(19) United States
(12) Patent Application Publication
(10) Pub. No.: US 2011/0097391 A1
(43) Pub. Date: Apr. 28, 2011

(54) Phospholipid Emulsion Containing Dihydroquercetin, and Method of Producing Thereof

(75) Inventors: Aleksej Mikhajlovich Grigor'ev, Moscow (RU); Anton Vladimirovich Evteev, Moskovskaya obl. (RU); Andrej Petrovich Smyslov, Moscow (RU); Pjotr Andreevich Smyslov, Moscow (RU); Andrej Petrovich Smyslov, Moscow (RU); Maksim Vjacheslavovich Cvetkov, Moskovskaya obl. (RU)

(73) Assignee: Obschestvo S Ogranichennoi Otvetstvennostju Scientific Company Flamena, Moskovskaya obl. (RU)

(21) Appl. No.: 12/982,175

(22) Filed: Dec. 30, 2010

Related U.S. Application Data
(63) Continuation of application No. PCT/RU2008/000819, filed on Dec. 31, 2008.

(51) Int. Cl. A61K 9/127; A61K 31/352; A61K 38/43; A61K 38/44; A61P 17/02

(52) U.S. Cl. 424/450; 514/456; 424/94.1; 424/94.4

(57) ABSTRACT

This present invention relates generally to the field of chemistry, food industry, cosmetics, and pharmaceutical industry. More specifically, the present invention relates to liposomal biologically active forms and methods of producing thereof. The present invention provides a stable phospholipid emulsion based on dihydroquercetin (DHQ), its compounds and derivatives. The invention further provides a method a quite simple and economic method for the preparation of disclosed emulsion. The disclosed emulsion is characterized by the improved bioavailabilty and selectivity of the effect of active compounds and by the increased stability and prolonged storage terms. The claimed result is achieved using a phospholipid emulsion containing DHQ, its compounds and/or derivatives, at least one membrane-forming phospholipid, at least one zwitter-ion amino acid base, aminoacetic ether, and aqueous ethanol in the following ratio of components (wt %): Dihydroquercetin, 0.01-3; Phospholipid, 0.15-10; Zwitter-ion amino acid base, 0-8; Aminoacetic ether, 0-4; Aqueous ethanol solution (with ethanol concentration within 0.05-20%), 75-98.84.

This present invention relates generally to the field of chemistry, food industry, cosmetics, and pharmaceutical industry. More specifically, the present invention relates to liposomal biologically active forms and methods of producing thereof.

In this field, many active substances are known, which are either slightly soluble or insoluble. Their practical use requires developing methods and means of delivery hydrophobic agents to water-containing media. Various systems of this kind have been developed, including mixtures of water with organic solvents, encapsulation, use of emulsions and suspensions, liposomes, and micelles. However, each of these systems has particular limitations related to the coefficient of trapping of the active agent, stability of this agent in the system, lifetime of the system, selectivity of delivery, etc.

For example, U.S. Pat. No. 4,776,991 (Farmer et al.) describes a wide-range encapsulation of hemoglobin using hydrophobic agents in liposomes. U.S. Pat. No. 5,010,073 (Kappas et al.) describes the preparation of liposomes containing metalloporphyrin with egg phosphatidylcholine (used as the lipid component). U.S. Pat. No. 5,922,355 (Parikh) describes microscopic particles containing insoluble components. Lasic [Nature, vol. 355, 379-380 (1992)] describes the use of aggregated micelles containing drugs and biological lipids.

In a similar manner, micelles have been used for the delivery of drugs during the therapy of patients (Brodin et al., Acta Pharm. Seuc. 19, 267-284). Micelles were also used for the targeted delivery of drugs [Supersaxo et al., Pharm. Res. 8:12864291 (1991)] including antitumor agents [(Fung et al., Biomater. Artif. Cells. Artif. Organs 16:439 et.seq. (1988) & Yokoyama et al., Cancer Res. 51:3229-3236 (1991)].

In U.S. Pat. No. 6,984,395 (Bok R. D. et al.), a phospholipid composition containing hydrophobic photosensitizers has been claimed. This composition can be converted into a stable liposomal product, which can be sterilized by ultrafiltration, lyophilized for storage, and then rapidly dissolved in an aqueous medium immediately prior to administration. Although some problems related to the stability and activity of the composition have been solved, this preparation technology is still rather complicated, additional operations and special conditions are necessary for the storage and for preparing the system to administration.

The present invention provides a stable phospholipid emulsion based on dihydroquercetin (DHQ), its compounds and derivatives. The invention further provides a method a quite simple and economic method for the preparation of this emulsion. The proposed emulsion is characterized by the improved bioavailabilty and selectivity of the active compounds and by the increased stability and prolonged storage terms.

The claimed result is achieved using a phospholipid emulsion containing DHQ, its compounds and/or derivatives, at least one membrane-forming phospholipid, at least one zwitter-ion amino acid base, aminoacetic ether, and aqueous ethanol in the following ratio of components (wt %):

### Dihydroquercetin, 0.01-3;

### Phospholipid, 0.15-10;

Zwitter-ion amino acid base, 0-8;
Aminoacetic ether, 0-4;
Aqueous ethanol solution
(with ethanol concentration within 0.05-20%), 75-98.84.

The effect of emulsion is determined by the properties of phospholipids and DHQ, as well as all other biologically active substances contained in liposomes. Each component, while performing its own function, naturally supplements the properties of other components and exhibits a synergistic action.

DHQ is a compound that belongs to the group of flavonoids, which produces angioprotector, regenerating, detoxicating, antiedematous, and antioxidant action. DHQ prevents premature aging of cells and the development of various disorders, reinforces vessel walls, improves microcirculation, suppresses inflammatory processes and edemation, favorably influences skin condition, normalizes the collagen/elastin synthesis, etc.

The main problem in using DHQ as a biologically active agent consists in the delivery of this compound to the site of potential action in the organism. DHQ is poorly soluble in water and highly sensitive to the presence of metal ions. This usually makes injections impossible, while peroral administration involves the risk of the loss of activity during passage via the gastrointestinal tract. p Phospholipids are the main structural components in all cellular membranes in the organism. This group includes, in particular, phosphatidylcholine (PC), phosphatidylethanolamine (PE) phosphatidylglycerol (PG), phosphatidyinositol (PI), and 1,3-diphosphatidylglycerol (cardiolipin). Due to the unique surfactant properties of phospholipids, the drugs containing these compounds are capable of restoring damaged parts of membranes and, hence, preventing the further development of structural cell pathologies. This therapy results in increasing osmotic resistance of cells (including erythrocytes), which is evidence for a “rejuvenation” of cells. Indeed, the cell walls become more elastic that is characteristic of young cells.

Despite the positive properties of phospholipids, there are objective factors hindering their practical usage. These include low solubility in water, high sensitivity to pH, and high reactivity (including the susceptibility to oxidation), which leads to problems in the administration and poses increased requirements on the conditions of storage and use.

The introduction of amino acids into the emulsion according to the present invention provides, in addition to the well-known positive biological effects, stability of the rheological parameters of the emulsion, preventing its premature separation into fractions. The stability of emulsion is ensured by zwitter-ion properties of the molecules of amino acids. In a preferred embodiment, the amino acid is glycine, but some other amino acids can also be used, these including glutamine, asparagine, alanine, valine, hnistidine, serine, isoleucone, threonine, praline, cysteine, lysine, tyrosine, phenylalanine, arginine, methionine, tryptophan, aspartic acid, glutamic acid, cystine, leucine, and their derivatives.

In order to impart the phospholipid emulsion additional biological properties, depending on a particular application field, various biologically active substances can be added such as polyene acids, enzymes (as well as their precursors and derivatives), antioxidants, vitamins (and their derivatives), minerals, UV-filters, preservatives, dyes, flavoring agents, and thickening components (at a total amount not exceeding 6%).

In a preferred embodiment, the polyene acid is selected from a group including linolenic acid, gamma-linolenic acid, dihomo-gamma-linolenic acid, arachidic acid (or their mixtures), while a vitamin are represented by vitamins A, E, and C. The antioxidants in the preferred embodiment are alpha-tocopherol, ubiquinone, and substances from the group of bioflavanoids; the preferred enzyme is 5,6 desaturase; the polyunsaturated fatty acid is represented by a mixture of C18:2 and C18:3 in a 1:1 ratio (Mol/ml). The emulsion may also contain hyaluronic acid (up to 3%) as the wetting agent.

The size of liposomes in the emulsion ranges from 20 to 500 nm, depending on the conditions of preparation. In all cases, it is possible to provide a high homogeneity of the emulsion with respect to the particle size distribution. In most widely used emulsions, about 70% of liposomes belong to the fraction with dimensions within 20-100 nm.

The liposomal organization of biologically active substances ensures the delivery of acting components to the target organs and tissues, allows the emulsion to retain its properties during storage under usual conditions and renders it resistant to oxidation. The emulsion retains preset composition and properties in a broad range of acidity (pH_3.0-10.0) and withstands heating to 65° C. for about one hour.

The introduction of DHQ into the composition of liposomes produces a double effect: (i) on the one hand, DHQ ensures active biological functioning of liposomes; (ii) on the other hand, DHQ (being a strong antioxidant) protects the phospholipids in liposomes against oxidation and ensures structural stability of the lipid bilayer. This is related to the method of including DHQ into liposomes. When the emulsion is prepared according to the claimed method, DHQ reacts with PL molecules and incorporates into the lipid bilayer. For this reason, the content of the active substance in liposomes is determined by the molar ratios of the initial components, rather than by the coefficient of trapping of the active agent.

The method of preparation of the phospholipid emulsion according to the present invention consists in

(i) preparing an extract, which represents a solution of DHQ and at least one phospholipid in ethanol, and a dispersion medium, which includes deionized water;

(ii) rotating the dispersion medium until the formation of a stable paraboloid of revolution (whirlpool);

(iii) introducing extract into the whirlpool;

(iv) separating the obtained mixture into a concentrate of the dispersed phase (liposomes) and the dispersion medium in a ratio of 1:2 to 15; and

(v) adding an aqueous solution of at least one zwitter-ion amino acid base to the concentrate.

During preparation of the extract (dispersed phase), alcohol-soluble biologically active substances, which are poorly soluble in water (e.g., DHQ, polyene acids, etc.) and are capable of interacting with a zwitter-ion base of a particular phospholipid (either directly or via a water-soluble mediator such as glycine also possessing a zwitter-ion base) are also introduced into the ethanol solution of phospholipids.

The disclosed method is analogous to the injection method used for the preparation of liposomes, according to which a dispersed phase (alcohol extract) is injected under pressure into a dispersion medium. However, the disclosed method takes into account the hydrodynamic characteristics of the dispersion medium during its stirring. Rotation of the stirrier leads to the formation of a whirlpool that is centered at the rotor. At a sufficiently high speed of rotation, a thin layer of aqueous dispersion medium is formed into which a dispersed phase is injected via a capillary. The formation of liposomes takes place in a thin layer over the rotor and, due to the centrifugal force, the liposomes are immediately redistributed over the entire volume of liquid. In this way, liposomes can be obtained at relatively small rotation speeds of 700-1500 rpm.

In preparing some cosmetics, it is necessary to remove the solvent (ethyl alcohol) from the final product, stabilize rheological properties of the preparation, and exclude phase separation of the suspension. Within the framework of the disclosed method, these tasks are readily solved by replacing the dispersion medium (aqueous-ethanol solution with a small content of finest fractions) after standing of the native solution by the aqueous glycine solution. Upon resuspension, this yields a stable solution, not susceptible to phase separation. Under certified storage conditions, the suspension retains its properties over several months (up to about one year).

The general description of the present invention having been made, a further understanding can be obtained by reference to particular examples, which are given herein only for the purpose of illustration and are not intended to limit the scope of the appended claims provided below.

### EXAMPLE 1

In order to prepare 1 liter of the phospholipid emulsion having a trade name Flamena D, 200 ml of extract are prepared as follows:

To a solution of 4 g DHQ (94-96% purity) in 166 ml of ethanol (96%) is added lecithin (30 g), preferably egg lecithin with a PC content of no less than 80%. The mixture is stirred until complete dissolution of lecithin. It is recommended to heat the mixture to a temperature of 50° C. to facilitate dissolution. The obtained solution can be stored at room temperature for several days.

The role of a dispersion medium is played by deionized distilled water (0.5 microsiemens). Same water is used to prepare a 5% glycine solution. The dispersion medium in a vessel (with a volume of up to 2 liter) is rotated on a magnetic stirrer at a rotation speed that is gradually increased to 700-1000 rpm. The thickness of a liquid layer at the center of the whirlpool must not exceed 10 mm. The extract prepared as described above is introduced into this site. The rate of extract introduction must be uniform, amounting to 3-4 ml/s.

After switching off the stirrer and allowing the mixture to stand until equilibration, a glycine solution is added at an amount necessary to fill the emulsion volume to 2000 ml.

In another embodiment, the mixture is separated into fractions, for example, by standing for 24 h at a reduced temperature (not exceeding 10° C.). As a result, two layers (phases) are formed: the bottom, dense white liquid accounting for 25-28 vol %, and the supernatant semitransparent liquid. The semitransparent phase is decanted. To the bottom phase is added a 5% glycine solution in deionized distilled water at an amount necessary to fill the emulsion volume to 2000 ml. Then, the product is stirred and poured into containers for storage and use.

The phospholipid emulsion according to the present invention was tested in cosmetics as a remedy accelerating skin repair, improving subcutaneous circulation, stabilizing exchange, etc. It was established that the claimed emulsion significantly accelerates recovery processes in the skin and improves the state of skin with respects to all the above criteria as compared to the control group, where the preparation was not applied.

A series of investigations were performed to study the effect of the phospholipid emulsion on the healing of wounds and burns under experimental conditions in vivo. The experiments were performed on white male rats. The emulsion was applied onto artificial wounds with aceptic inflammation, which were formed on depilated areas on the back of animals under ether narcosis. Full-depth wounds with nonpurulent (aceptic) inflammation were modeled by excising skin to fascia on an area with a diameter of about 2.5 cm. Then, the wounds were treated using dressings of various compositions. Depending on the type of treatment, the experimental animals were divided into three groups as follows:

(1) Gauze dressing with Flamena D emulsion (first test group);

(2) Gauze dressing with DHQ solution (second test group);

(3) Gauze dressing with physiological solution (control group).

The course of the wound process was monitored based using the following criteria: (1) results of clinical examination of the character of the wound process, including

the presence (absence) of edemation on the wound body and edges, hyperemia, and painful sensation during manipulations;
the presence (absence) of a necrotic detritus and exudation;
terms of wound cleansing from purulent-necrotic detritus, relieve of inflammation, formation and development of granulation tissue, and the onset of epithelization;
(2) objective clinical criteria of the wound process development, including
planimetric measurements for evaluating a decrease in the wound area;
dynamic morphological analysis of wound bioptates;
results of microbiologial analysis.

Based on an analysis of the results of clinical observations after the wound process in experimental animals and the efficacy of treatment with Flamena D and DHQ, the following was established.

(1) In comparative aspect, Flamena D emulsion produces a more favorable effect on the wound process as compared to that of DHQ solution and physiological solution (control group), which is manifested by an insignificant aceptic inflammation in the early stage of the wound process in animals treated with Flamena D;
(2) Clinical data are reliably indicative of the favorable effect of Flamena D emulsion on the angiogenesis at all terms of the observation of the state of model wounds;
(3) An analysis of the results of treatment of the model wounds shows that Flamena D emulsion produces pronounced anti-inflammatory and stimulating effect on the angiogenesis and the growth of granulation tissue, which ensures the efficacy of treatment in terms of the inflammation arrest, development of the skin repair process, and the dynamics of wound defect reduction.

The results of morphological investigation also showed efficacy of the application of Flamena D emulsion forte treatment of skin wounds. The dressing is most effective at the end of stage I or at the beginning of stage II of the wound process (i.e., within 2-5 days). Flamena D emulsion stimulates the angiogenesis process and fibroblast proliferation more effectively than DHQ solution with respect to both morphological manifestations and quantitative analysis. As a result of the application of Flamena D dressing, the degree of granulation tissue maturity was much greater than that in the case of treatment with DHQ solution.

The effect of Flamena D was also experimentally studied in vivo on animals with chemical and thermal skin burns. For this purpose, model chemical and thermal bums of degree 2 were formed on the skin of animals under ether narcosis. Then, the burns were treated by periodic application of Flamena D emulsion. The results were compared to the control group, where the animals were treated with physiological solution. The efficacy of the treatment was evaluated by the results of morphological and histological investigations. The results of these tests showed that the treatment with Flamena D emulsion significantly accelerates the process of model wound healing in the experimental animals. In addition, this treatment also favorably influenced the process of skin repair, which was manifested by the absence of cicatrix and by faster recovery of the wool.

1. A phospholipid emulsion comprising:
dihydroquercetin, its compounds and/or derivatives, at least one membrane-forming phospholipid , at least one zwitter-ion amino acid base, aminoacetic ether, and aqueous ethanol in the following ratio of components (wt %): Dihydroquercetin , 0.01-3;
Phospholipid, 0.15-10;
Zwitter-ion amino acid base, 0-8;
Aminoacetic ether, 0-4; and
Aqueous ethanol solution
(with ethanol concentration within 0.05-20%), 75-98.84.

2. A phospholipid emulsion according to claim 1, wherein dihydroquercetin is contained in liposome walls.

3. A phospholipid emulsion according to claim 1, wherein said phospholipids is selected from the group including phosphatidylcholine (PC), phosphatidylethanolamine (PE) phosphatidylglycerol (PG), phosphatidyinositol (PI), and 1,3-diphosphatidylglycerol (cardiolipin) or their mixtures.

4. A phospholipid emulsion according to claim 1, wherein said zwitter-ion amino acid base is represented by substances from the group including glutamine, asparagine, alanine, valine, hnistidine, serine, isoleucone, threonine, praline, cysteine, lysine, tyrosine, phenylalanine, arginine, methionine, tryptophan, aspartic acid, glutamic acid, cystine, leucine, and their derivatives.

5. A phospholipid emulsion according to claim 1, further comprising up to 6% of substances selected from the group including polyene acids, enzymes (as well as their precursors and derivatives), antioxidants, vitamins (and their derivatives), minerals, UV-filters, preservatives, dyes, flavoring agents, and thickening agents.

6. A phospholipid emulsion according to claim 5, wherein said polyene acids being selected from a group including linolenic acid, gamma-linolenic acid, dihomo-gamma-linolenic acid, arachidic acid or their mixtures.

7. A phospholipid emulsion according to claim 5, wherein said vitamins are selected from the group including vitamins A, B₅ C, E, K, F, and D.

8. A phospholipid emulsion according to claim 5, wherein said antioxidants are selected from the group including alpha-tocopherol, ubiquinone, and substances from the group of biofiavanoids.

9. A phospholipid emulsion according to claim 5, wherein said enzyme is 5,6 desaturase.

10. A phospholipid emulsion according to claim 5, further comprising a polyunsaturated fatty acid representing a mixture of C18:2 and C18:3 in a 1:1 ratio (Mol/ml).

11. A phospholipid emulsion according to claim 1, further comprising up to 3% of hyaluronic acid.

12. A phospholipid emulsion according to claim 1, wherein the size of liposomes ranges from 20 to 500 run.

13. A phospholipid emulsion according to claim 1, wherein about 70% of liposomes belong to a fraction with dimensions within 20-100 nm.

14. A phospholipid emulsion according to claim 1, wherein the acidity varies within pH of 3.0-10.0.

15. A method of preparation of the phospholipid emulsion, comprising:
(i) preparing an extract, which represents a solution of dihydroquercetin and at least one phospholipid in ethanol, and a dispersion medium, which contains deionized water;
(ii) rotating the dispersion medium until the formation of a stable whirlpool;
(iii) introducing extract into the whirlpool;
(iv) separating the obtained mixture into a concentrate of the dispersed phase and the dispersion medium in a ratio of 1:2 to 15; and
(v) adding an aqueous solution of at least one zwitter-ion amino acid base to the concentrate.

16. The method of preparation of the phospholipid emulsion according to claim 15, wherein said dispersion medium comprises aminoacetic ether.

17. The method of preparation of the phospholipid emulsion according to claim 15, wherein the dispersion medium is rotated using a magnetic stirrer at a rotor speed of 700-1500 rpm.

18. The method of preparation of the phospholipid emulsion according to claim 15, wherein the extract is introduced at the center of the whirlpool.

19. The method of preparation of the phospholipid emulsion according to claim 15, wherein the obtained mixture is allowed to stand at a temperature of 0-20° C.

20. The method of preparation of the phospholipid emulsion according to claim 15, wherein the obtained mixture is concentrated by separating into fractions.
